(19) United States
(12) Patent Application Publication
(10) Pub. No.: US 2010/0094358 A1
(43) Pub. Date: Apr. 15, 2010

(54) SPINAL STAPLE

(75) Inventors: Brandon Moore, Summit Point, WV (US); Oheneba Boachie-Adjei, Briarcliff, NY (US); Jennifer Haggenmaker, Summit Point, WV (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/578,259

(22) Filed: Oct. 13, 2009

Related U.S. Application Data
(60) Provisional application No. 61/195,826, filed on Oct. 10, 2008.
(60) Provisional application No. 61/184,045, filed on Jun. 4, 2009.

(51) Int. Cl. A61B 17/86; A61B 17/064

(52) U.S. Cl. 606/319; 606/75

(57) ABSTRACT

A bone anchor assembly includes a bone fixation member and a bone screw. The bone fixation member includes at least one opening therethrough for receiving the bone screw. Two pairs of legs extend from a plate portion of the bone fixation member and are configured for insertion into bone. A lip of the opening is configured for engaging a set of threads on the bone screw such that the bone screw and the bone fixation member are affixed to one another once the bone screw is inserted through the opening of the plate portion. An installation tool for inserting the bone fixation member into bone includes a proximal region, a tubular member, a rotatable collar, and a gripping mechanism.

### CROSS-REFERENCE TO RELATED APPLICATIONS

This application claims priority to, and the benefit of, U.S. Provisional Patent Application Ser. No. 61/184,045, filed Jun. 4, 2009, and U.S. Provisional Patent Application Ser. No. 61/195,826, filed Oct. 10, 2008. The disclosures of these prior applications arc hereby incorporated by reference in their entirety.

### BACKGROUND

1. Technical Field

The present disclosure relates to devices for implantation on the exterior of adjacent vertebrae. Specifically, the disclosure relates to a spinal staple that corrects deformities and can be used in conjunction with an interbody spacer device to stabilize the spine after a discectomy.

2. Background of Related Art

After a partial or complete discectomy, the normally occupied space between adjacent vertebral bodies is subject to collapse and/or misalignment due to the absence of all or a part of the intervertebral disc. In such situations, the physician may insert one or more prosthetic spacers between the affected vertebrae to maintain normal disc spacing and/or the normal amount of lordosis in the affected region.

Typically, a prosthetic implant is inserted between the adjacent vertebrae and may include pathways that permit bone growth between the adjacent vertebrae until they are fused together. However, there exists a possibility that conventional prosthetic implants may be dislodged or moved from their desired implantation location clue to movement by the patient before sufficient bone growth has occurred.

Therefore, a need exists for a spinal staple that provides support to the interbody spacer and helps resist dislocation from the implantation site as well as help to maintain the lordosis or kyphosis.

### SUMMARY

According to one embodiment of the present disclosure, a spinal staple or bone fixation member for engagement on the exterior of vertebrae includes a body having a first end surface at a distal end of the body and a second end surface opposite thereto at a proximal end of the body. The body extends between the first and second end surfaces to define opposing top and bottom vertebral engaging surfaces substantially symmetrical about a centerline axis. The body further defines side surfaces. There are legs on the bottom surface of the staple that allow for engagement in the vertebral body. The top surface of the body includes at least one opening formed therethrough. The bone fixation member has a screw opening defined therethrough and is configured to be mounted to the body with the bone screw opening substantially aligned with the at least one opening. The opening is configured with a lip disposed in the screw opening configured to engage threads of a bone screw to secure the bone screw within the at least one bone fixation member.

An installation tool for use with the presently disclosed bone fixation member is also disclosed. The installation tool includes a proximal region having a hardened surface for engaging a striking tool (e.g. a hammer), a tubular member extending from the proximal region, a rotatable collar, and a gripping mechanism. Rotating the rotatable collar transitions fingers of the gripping mechanism between first and second conditions for engaging or releasing the bone fixation member.

In one embodiment, the installation tool further includes a central shaft having a sharpened distal tip for positioning the installation tool on a bone. The installation tool may also be used with a coupling member adapted for use with bone fixation members having more than one opening.

In one embodiment, the bone fixation device includes a circular central opening for cooperation with an installation tool. In a further embodiment, the central opening is elongated.

### BRIEF DESCRIPTION OF THE DRAWINGS

Embodiments of the presently disclosed spinal staple are described herein with reference to the accompanying drawings, wherein:

FIG. 1 is a perspective view of a spinal staple in accordance with an embodiment of the present disclosure;

FIG. 2A is a top plan view of the spinal staple of FIG. 1;

FIG. 2B is a side cross-sectional view of the spinal staple of FIG. 2A taken along section line 2B-2B;

FIG. 3 is an alternate embodiment of a spinal staple in accordance with the present disclosure;

FIG. 4 is a perspective view of a bone fixation assembly including the spinal staple of FIG. 1 and a pair of bone screws coupled thereto;

FIG. 4A is a side view of the bone fixation assembly of FIG. 4;

FIG. 5 is a perspective view of the spinal staple of FIG. 3 with a bone screw coupled thereto;

FIG. 6 is a perspective view of an installation instrument and the spinal staple of FIG. 3;

FIG. 7 is a perspective view of the installation instrument of FIG. 6 with an adapter for use with the spinal staple of FIG. 1;

FIG. 8A is a perspective view of an alternate embodiment of the spinal staple in accordance with the present disclosure;

FIG. 8B is a bottom plan view of the spinal staple of FIG. 8A; and

FIG. 9 is a perspective view of an installation instrument for use with the spinal staple of FIG. 8A.

### DETAILED DESCRIPTION

Various embodiments of the presently disclosed spinal staple will now be described in detail with reference to the drawings, wherein like reference numerals identify similar or identical elements. In the drawings and in the description that follows, the term “proximal,” will refer to the end of a device or system that is closest to the operator, while the term “distal” will refer to the end of the device or system that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e. away from the middle of the body of the patient). The term “posterior” indicates a direction toward the patient's back, and the term “anterior” indicates a direction toward the patient's front. Additionally, in the drawings and in the description that follows, terms such as front, rear, upper, lower, top, bottom, and the similar directional terms are used simply for convenience of description and are not intended to limit the disclosure attached hereto.

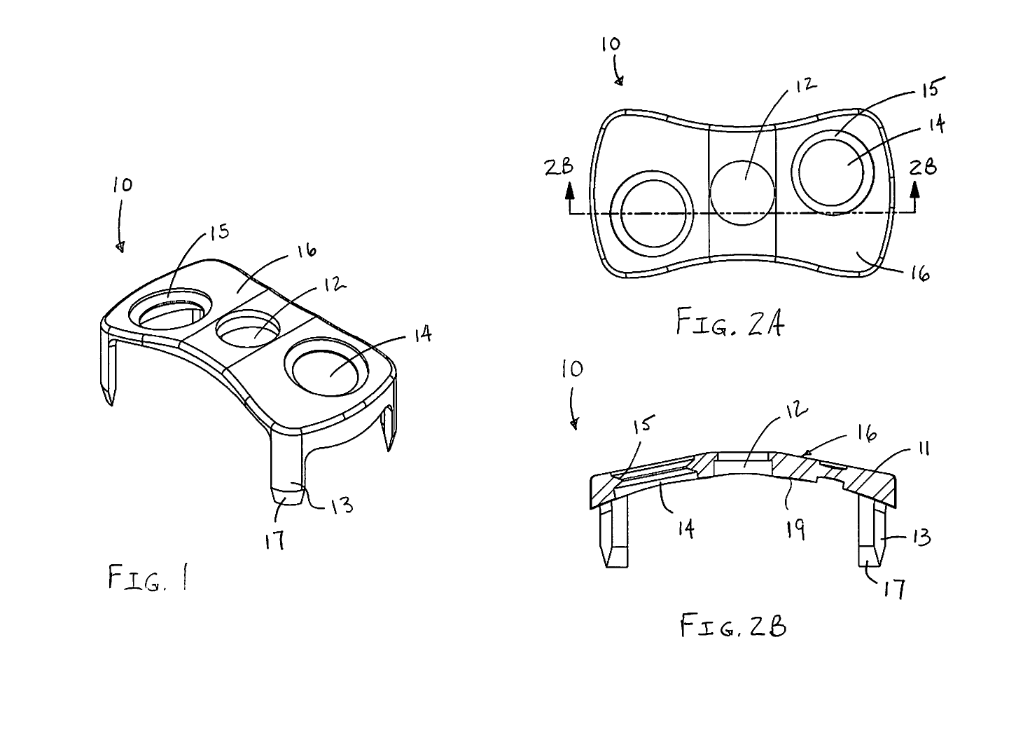

Referring to FIGS. 1, 2A, 2B, and 4, a bone anchor assembly 100 includes a bone fixation member 10 and at least one screw 50. As shown in the accompanying figures, the screw 50 is a taper lock style screw. It is within the scope of the present disclosure that set screw style screws may be substituted for the illustrated taper lock style screws without departing from the scope of the present disclosure. An example of suitable taper lock style screw is disclosed in International Patent Application Number PCTUS/2008/080682, filed Oct. 22, 2008 and published as WO 2009/055407 A1, the entire contents of which are hereby incorporated by reference. Further, an example of a suitable set screw style screw is disclosed in International Patent Application Number PCTUS/2008/080668, filed on Oct. 22, 2008 and published as WO 2009/055400 A1, the entire contents of which are hereby incorporated by reference.

The bone fixation member 10 includes a plate portion 16 having a central orifice 12 and an opposed pair of openings 14. The bone fixation member 10 is a low profile device that is securable to a bone structure (e.g. a vertebra) in a patient using one or more screws 50. Each of the openings 14 includes a lip 15. Although the openings 14 are illustrated as being generally circular, it is contemplated that they may be elongated openings. In one embodiment, the plate portion 16 is formed from a relative soft material such as commercially pure titanium. The relatively soft material of the plate portion 16, and the lip 15, cooperates with the screw 50 such that the screw 50 is secured to the bone fixation member 10 as will be discussed in further detail hereinafter. A pair of legs 13 is disposed at a first end of the plate portion 16 and a second pair of legs 13 is disposed at a second end of the plate portion 16. Each leg includes a distal tip 17 configured for insertion into bone. In one embodiment, the distal tip 17 has a chisel point, although other suitable configurations arc contemplated and are within the scope of the present disclosure. Further still, the distal tips 17 may be hardened, as is known in the art, for facilitating penetration into bone.

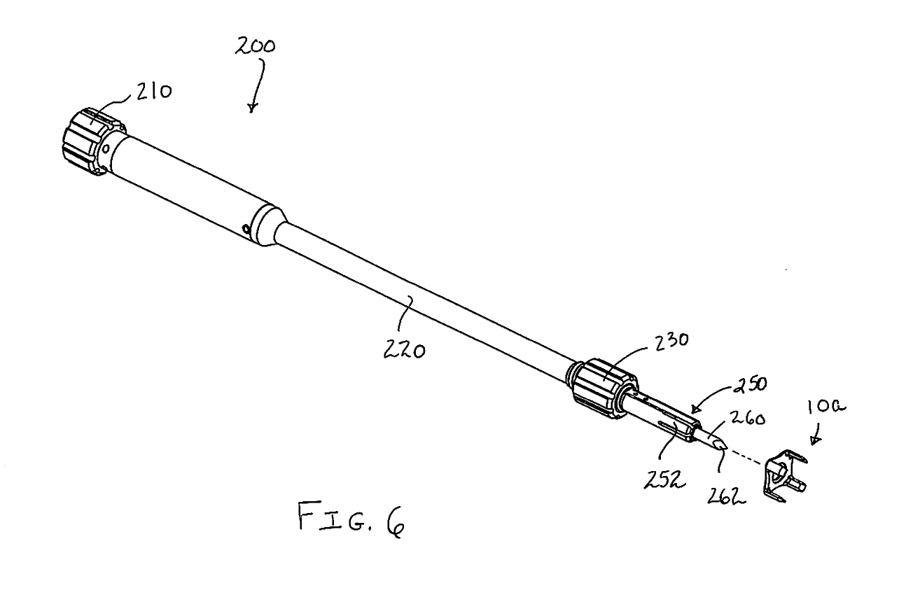

With particular attention to FIG. 4, the bone screw 50 includes a head portion 60 and a shank 40 extending therefrom. The head portion 60 includes a housing 62 and a collet 64. The collet 64 includes a generally U-shaped saddle 66 for releasably receiving a spine rod therein. The collet 64 and the housing 62 are pivotable and rotatable relative to the shank 40. After a rod (not shown) is positioned in the U-shaped saddle 66, relative movement between the housing 62 and the collet 64 secures the rod to the bone screw 50. Referring additionally to FIG. 4A, the shank 40 includes first threads 42 located near the head of the bone screw 50 and second threads 44 formed on an outer surface of the shank 40. The second threads 44 are configured and adapted for penetration into bone. The first threads 42 are arranged such that when the bone screw 50 is inserted through the opening 14, the first threads 42 engage the lip 15 of the opening 14. In one embodiment, the bone screw 50 is formed from a relatively hard material such as Ti-6A1-4V, which is a titanium alloy. Since the first threads 42 are formed of a harder material than the lip 15, the first threads 42 deform the lip 15 as the bone screw 50 advanced through the opening 14 of the bone fixation member 10. The pitch of the first threads 42 is substantially uniform throughout. Similarly, the pitch of the second threads 44 is also substantially uniform throughout, although the pitch of the first threads 42 may be different from the pitch of the second threads 44.

In particular, since the plate portion 16 is formed from a material that is softer than the material of the first threads 42, when the screw 50 is inserted through the opening 14, the first threads 42 deform the lip 15 of the opening 14, thereby securing the bone screw 50 and plate portion 16 to each other. In this arrangement, the bone screw 50 resists backing out of the opening 14. This type of screw locking arrangement is disclosed and shown in U.S. Pat. No. 6,322,562 to Wolter, the entire contents of which are hereby incorporated by reference herein.

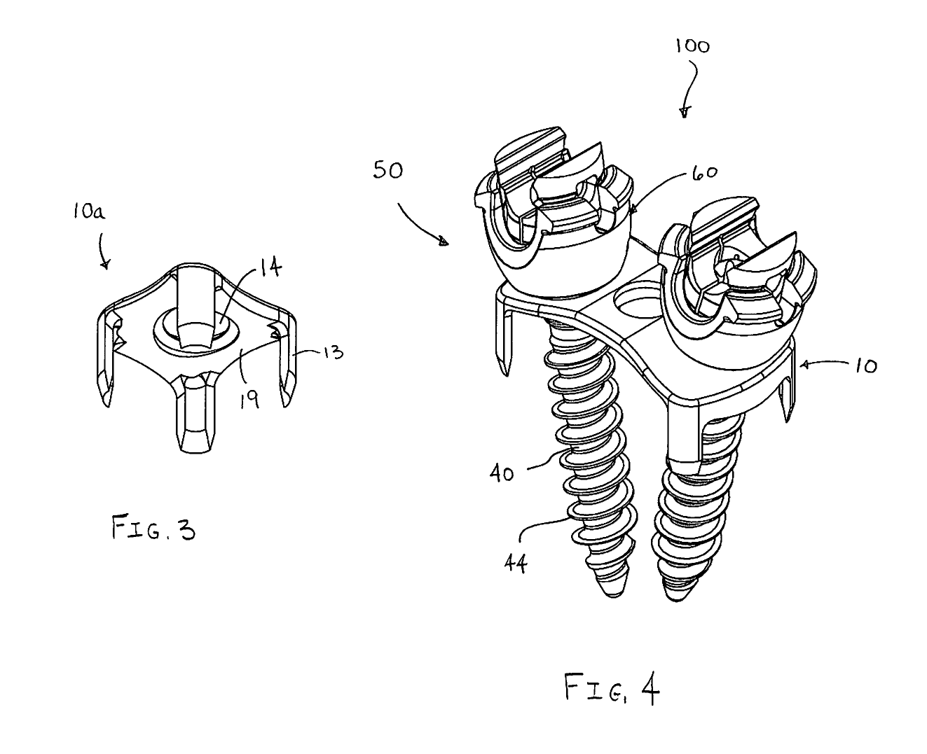

Referring again to FIGS. 1, 2A, and 2B, the plate portion 16 of the bone fixation member 10 has an arcuate upper surface 11 and an arcuate lower surface 19. The upper and lower surfaces 11, 19, are configured for substantially flush engagement with a vertebral body of a patient.

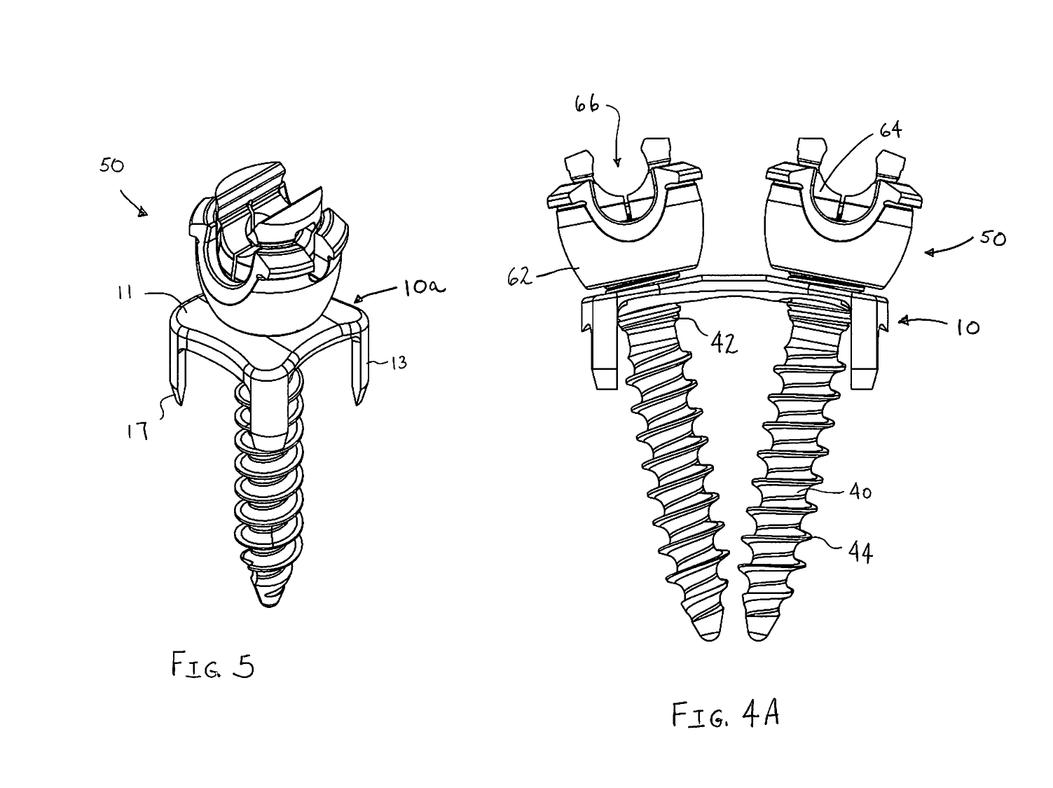

Referring now to FIGS. 3 and 5, an alternate embodiment of the presently disclosed bone fixation member is shown and generally referenced as 10a. Bone fixation member 10a is substantially similar to bone fixation member 10, with the differences discussed in detail hereinbelow. Unlike bone fixation member 10, bone fixation member 10a includes a single opening 14 with a lip 15. In FIG. 5, the bone fixation member 10a is shown with the bone screw 50 and forms bone anchor assembly 100a.

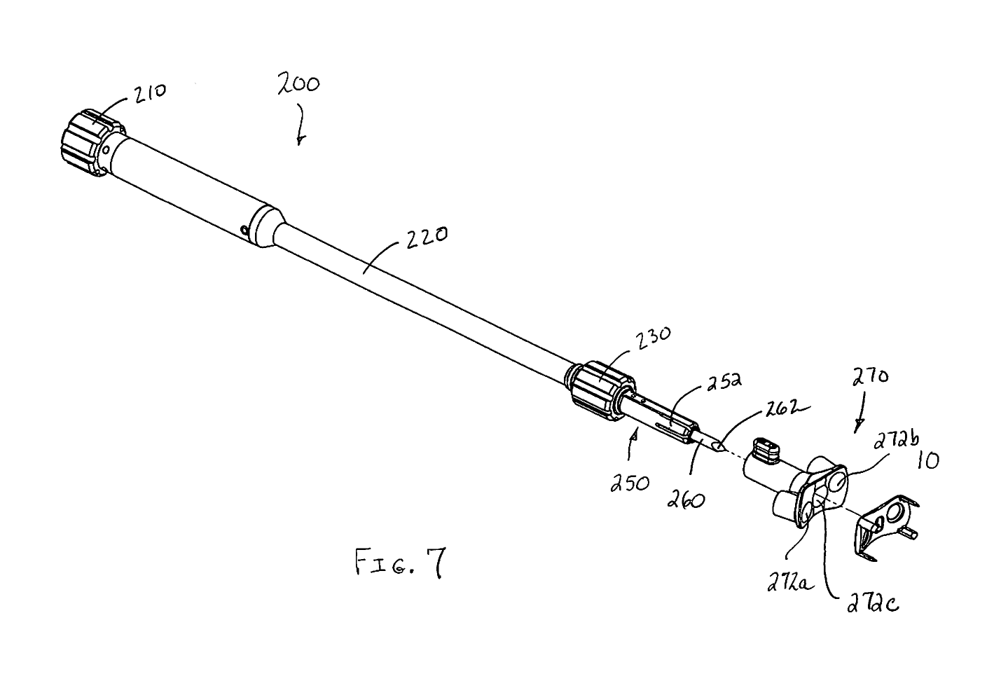

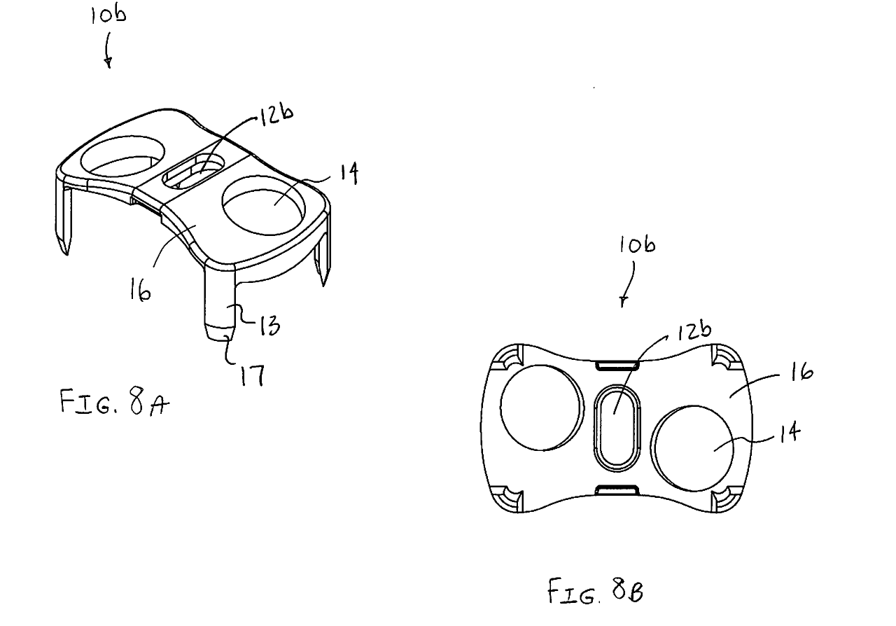

FIGS. 6 and 7 illustrate a tool 200 for inserting bone fixation members 10 and 10a, respectively, into bone. The tool 200 includes a hardened proximal end region 210, a tubular member 220, a rotatable collar 230, a gripping mechanism 250, and a central shaft 260. With initial reference to FIG. 6, the central shaft 260 includes a pointed distal end 262 that is configured to form a depression into a bone structure and assist in aligning the tool 200 to a desired location. The central shaft 220 is sized for insertion through the opening 14 of bone fixation member 10a. The gripping mechanism 250 includes a plurality of fingers 252 that are deflectable upon rotation of the collar 230. The gripping mechanism 250 is also sized to fit through the opening 14 when in a first state. Rotation of the collar 230 in a first direction spreads the fingers 252 such that they frictionally engage an inner surface of the opening 14, thereby securing the bone fixation member 10a to the tool. Rotating the collar 230 in the opposing direction retracts the fingers 252 such that they are released from the opening 14. Once assembled, a hammer or other suitable striking device is used to strike against a surface of the hardened proximal region 210. This drives the pointed distal end 262 and the distal tips 17 of the legs into bone, thereby securing the bone fixation member 10a in bone. Once the bone fixation member 10a is installed in bone, the bone screw 50 is inserted through the opening 40 and affixed to the bone using the first threads 42, while the second threads 44 affix the bone screw 50 to the bone fixation member 10a as fully described hereinabove.

In FIG. 7, the tool 200 is coupled to the bone fixation member 10 using a coupling member 270. The coupling member 270 includes orifices 272a-c that correspond to the openings 14 and the central orifice 12 of the bone fixation member 10. The outer orifices 272a, 272b may be angled for positioning the bone screws 50 at a desired angle for increased purchase in the underlying bone structure. The central orifice 272c is aligned with a central tube 274 that slidably receives the gripping mechanism 250 of the tool. The assembly of the tool 200 and the bone fixation member 10 is substantially similar to that of the tool 200 and the bone fixation member 10a described in detail hereinabove. However, instead of the fingers 252 frictionally engaging or releasing from the inner surface of the opening 14, the fingers 252 frictionally engage or release from an inner surface of the central orifice 12.

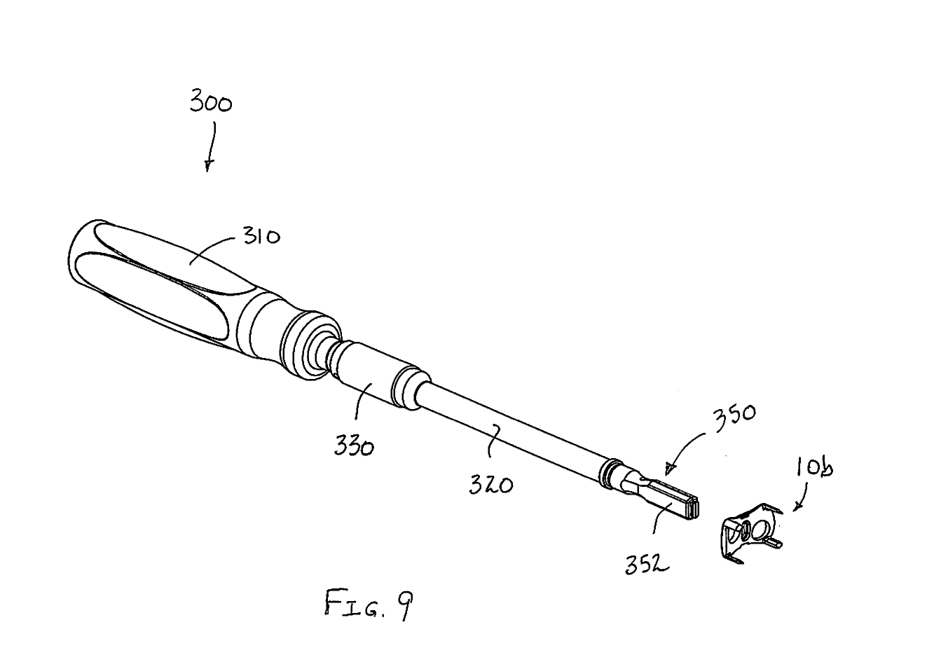

In an alternative embodiment illustrated in FIGS. 8A and 8B, a bone fixation member 10b is substantially identical to bone fixation member 10, but includes an elongated central orifice 12b. The elongated central orifice 12b is configured and adapted to cooperate with an installation tool 300 shown in FIG. 9. The installation tool 300 includes a handle portion 310 with a hardened proximal end, a tubular member 320, a rotatable collar 330, and a gripping mechanism 350. The hardened proximal end of handle portion 310 is configured and adapted for receiving striking blows from a hammer (not shown) or other suitable striking tool. The gripping mechanism 350 includes a pair of fingers 352 that are repositionable between a first state and a second state. When the fingers 252 are in the first state, the installation tool 300 is insertable into the central orifice 12b. Rotation of the collar 330 in a first direction spreads the fingers 352 apart such that they frictionally engage an inner surface of the orifice 12b, thereby securing the bone fixation member 10b to the installation tool 300. When the bone fixation member 10b is positioned in a desired location, the physician uses a hammer (not shown) or another suitable striking tool to drive the bone fixation member 10b into bone. Once inserted into bone, the bone fixation member 10b is released from the installation tool 300 by rotating the collar 330 in the opposing direction causing the fingers 352 to move towards each other, thereby releasing the tool 300 from the bone fixation member 10b. Subsequently, the physician may install a bone screw 50 through the openings 14 as described in detail hereinabove with respect to the bone fixation members 10, 10a.

Once the desired number and location of bone anchor assemblies are installed, the physician may then install one or more spine rods and lock them to the bone anchor assemblies to complete the rod and screw construct.

The above-described method of use can be employed as a method of stabilizing or fixing injured or diseased vertebrae and if necessary, multiple devices or a device, which is elongated beyond the examples depicted herein, can be employed as necessary.

While the bone fixation members as described herein can be used to attach to the anterior surface of cervical vertebrae and is configured to be capable of stabilizing cervical vertebrae, it is within the scope of the present disclosure that the bone fixation members can be configured and adapted to conform to any requirement to provide a low profile bone fixation member capable of securing and stabilizing any injured or diseased bone.

The presently disclosed bone fixation members and bone screws can be manufactured by methods known in the art, to include, for example, molding, casting, forming or extruding, and machining processes. The components can be manufactured using materials having sufficient strength, resiliency, and biocompatibility as is well known in the art for such devices. By way of example only, suitable materials can include implant grade metallic materials, such as titanium, cobalt chromium alloys, stainless steel, or other suitable materials for this purpose. It is also conceivable that some components of the device can be made from plastics, composite materials, and the like. Further, the surface of the bone fixation members may be anodized to provide a porous coating for absorbing a colored dye and/or to provide corrosion resistance.

It is also within the scope of the present disclosure to provide a kit, which includes at least one of the bone fixation members and a number of bone screws. The kit can also include additional orthopedic devices and instruments; such as for example, instruments for tightening or loosening the bone screws, spinal rods, hooks or links and any additional instruments or tools associated therewith. Such a kit can be provided with sterile packaging to facilitate opening and immediate use in an operating room.

Each of the embodiments described above are provided for illustrative purposes only. It will be understood that various modifications may be made to the embodiments of the presently disclosed vertebral body staple. Therefore, the above description should not be construed as limiting, but merely as exemplifications of embodiments. Those skilled in the art will envision other modifications within the scope and spirit of the present disclosure.

What is claimed is:

1. An anchor assembly comprising:
a bone fixation member having a plate portion including,
an opening having a lip,
a first pair of legs disposed at a first end thereof, and
a second pair of legs disposed at a second end thereof, wherein each leg includes a distal tip configured for penetration into bone; and
a screw having a head and a shank extending therefrom, the shank having threads formed on an outer surface thereof, wherein engagement between the threads of the screw and the lip of the opening deform the lip thereby affixing the screw to the bone fixation member.

2. The anchor assembly of claim 1, wherein the screw includes a U-shaped channel for receiving a rod.

3. The anchor assembly of claim 1, wherein the screw includes threads for engaging a set screw.

4. The anchor assembly of claim 1, wherein the screw includes a coupling and a collet such that relative movement between the coupling and the collet releasably retain a rod with the screw.

5. The anchor assembly of claim 1, wherein the plate portion is formed from a first material and the screw is formed from a second material that is harder than the first material.

6. The anchor assembly of claim 5, wherein the plate portion is formed from titanium.

7. The anchor assembly of claim 5, wherein the screw is formed from a titanium alloy.

8. The anchor assembly of claim 1, wherein the bone fixation member is releasably attachable to an installation tool.

9. The anchor assembly of claim 1, wherein the bone fixation member includes a plurality of openings.
